(19) United States
(12) Patent Application Publication
(10) Pub. No.: US 2026/0157976 A1
(43) Pub. Date: Jun. 11, 2026

(54) DICLOFENAC-CONTAINING TTS COMPRISING DIMETHYLPROPYLENEUREA

(71) Applicant: LTS Lohmann Therapie-Systeme AG, Andernach (DE)

(72) Inventors: Horst DZEKAN, Meinborn (DE); Luisa WITTLICH, Frankfurt am Main (DE); Nico REUM, Mendig (DE)

(21) Appl. No.: 18/707,002

(22) PCT Filed: Nov. 4, 2022
(86) PCT No.: PCT/EP2022/080881

§ 371 Date: May 2, 2024

(30) Foreign Application Priority Data
Nov. 5, 2021 (DE) 10 2021 128 911.4

(51) Int. Cl. A61K 9/70; A61K 31/196; A61K 47/10; A61K 47/12; A61K 47/22

(52) U.S. Cl. CPC A61K 9/7053; A61K 31/196; A61K 47/10; A61K 47/12; A61K 47/22

(57) ABSTRACT

The present invention relates to a transdermal therapeutic system for administering of diclofenac comprising a backing layer and a matrix layer, the matrix layer containing diclofenac, at least one occlusive adhesive component and at least one solvent, characterized in that the at least one solvent comprises dimethylpropyleneurea, and to the use thereof as a medicinal product.

The invention relates to a transdermal therapeutic system or a transdermal patch containing an active ingredient for the administration of diclofenac (2-[2-[(2,6-dichlorophenyl)amino]phenyl]acetic acid). The invention further relates to such a system for use as a medicinal product, more particularly for use in pain and inflammatory conditions.

Transdermal therapeutic systems are a dosage form for administering drugs in form of a patch. These systems have certain advantages over conventional dosage forms. When the patch containing the active ingredient is applied on the skin, said active ingredient will be absorbed through the skin of the corresponding part of the body exactly in this area and in the exact dose, without premature degradation in the gastrointestinal tract or liver. In addition, this dosage form enables a constant release of the active ingredient over a longer period of time.

In many cases, the transdermal administration of active ingredients is hampered by the low permeability of the skin. It was therefore important to increase the permeability of the skin for an efficient absorption of active ingredients. One possibility for this is the effect of occlusion, which is understood to mean a covering or sealing of a skin area to the maximum extent possible causing an accumulation of water vapor in the upper layers of the skin which, in turn, causes a higher permeability of the skin in relation to the active ingredient.

However, these well-known patches have the drawback, that their material properties generally are rather inelastic and rigid, which makes them less comfortable to wear, thus limiting the wearer's mobility and causing the patch to come off frequently and unintentionally.

According to DE 10103860 A1, the occlusion of the patch is achieved by using a backing layer that is impermeable to water vapor, such as a thin plastic film, preferably a polyethylene terephthalate (PET) film.

Another possibility to increase the permeability of the skin for the absorption of an active ingredient is the use of penetration enhancers.

EP 1 312 360 A1 discloses an analgesic, anti-inflammatory patch (“Dojin Patch”) for the local release of diclofenac. The system includes N-methyl-2-pyrrolidone as a solvent and thus ensures increased permeation of the active ingredient through the skin. The drawback of this system is that this solvent is a substance of concern for human health, and according to ICH guideline Q3C (dated Feb. 4, 2011), a daily intake of 5.3 mg N-methyl-2-pyrrolidone should not be exceeded.

The object of the present invention is to remove the above-mentioned drawbacks of the prior art. More particularly, the object of the present invention is to provide a transdermal therapeutic system which produces an optimal absorption of an active ingredient via the skin, and at the same time does not require the solvent N-methyl-2-pyrrolidone, which is harmful to health. Additionally, the system should be occlusive, and yet offer a high level of comfort and adhesion, even on flexible parts of the body such as joints.

The object described above is solved by a transdermal therapeutic system according to claim 1, comprising a backing layer and a matrix layer, wherein the matrix layer contains diclofenac, at least one occlusive adhesive component and at least one solvent, and being characterized in that the at least one solvent comprises dimethylpropyleneurea.

When describing the transdermal therapeutic system according to the invention, the term “comprising” may also mean “consisting of”.

The transdermal therapeutic system according to the invention is further preferably characterized in that the matrix layer as a whole is occlusive.

It is generally possible for the matrix layer to be formed as a single layer, i.e. the occlusive adhesive component is contained in this single-layer matrix layer and ensures that the system as a whole is occlusive.

Occlusion refers to the covering or sealing of skin regions, at least to the maximum extent possible, with materials that are impermeable to water vapor. As a result, the insensible perspiration (Perspiratio insensibiis, water or water vapor release through the skin of a person at rest) is impaired, leading to an accumulation of moisture, and consequently to hydration of the stratum corneum (outermost layer of the epidermis). Under occlusive conditions, the water content of the stratum corneum increases by up to 25% (m/m), preferably by up to 50% (m/m). The surface temperature of the skin can also rise to 37° C. Preferably, the amount of water vapor released is less than 500 g/m², particularly preferably less than 200 g/m² within 24 h, measured according to DIN EN 13726-2:2002 at a temperature of 37° C. and a relative humidity of 30%.

An adhesive component is understood as a substance that is either an adhesive in itself, preferably a pressure-sensitive adhesive, i.e. is tacky in itself, or produces an adhesive when mixed with other substances. An adhesive is a substance which, as defined in DIN EN 923 (June 2008), is a non-metallic substance capable of joining parts by surface bonding (adhesion) and adequate internal strength (cohesion). A substance or mixture of substances is described as tacky if it can be used as an adhesive in itself, more particularly as a pressure-sensitive adhesive. Pressure-sensitive adhesives are adhesives that remain highly viscous and permanently tacky after being applied to a carrier material and can then be applied to a substrate by applying light pressure and remain stuck. Pressure-sensitive adhesives, as defined in DIN EN 923 (June 2008), are also characterized by the fact that their set, dry film is permanently tacky at room temperature, as defined in DIN EN 923 (June 2008), and remains adhesive. Adhesive bonds produced with pressure-sensitive adhesives can usually be removed without destroying the bonded substrates.

The at least one occlusive adhesive component preferably comprises a pressure-sensitive adhesive or produces a pressure-sensitive adhesive by mixing with other substances.

The at least one occlusive adhesive component is thus to be understood to be a component that largely prevents insensible perspiration, i.e. the loss of water vapor from the skin, and thus leads to an accumulation of moisture in the stratum corneum. Preferably, the at least one occlusive adhesive component is an occlusive adhesive component that is already inherently tacky.

Permeability is the ability of solids (including porous solids), especially thin partitions, to let pass certain substances (gases, liquids, dissolved molecules, ions, or atoms). In the present case, the ability of human or animal skin to let pass small molecules, more particularly active pharmaceutical ingredients. In technical terms, permeation is understood to be the process of one substance passing through or penetrating another one. The term is often used in the context of the penetration of cosmetic or active pharmaceutical ingredients into or through the skin.

A penetration enhancer thus is understood to be a compound that enhances the permeation of one substance through another one, i.e. increases the permeation rate or generally increases the efficiency of permeation.

In addition, the at least one penetration enhancer is preferably a compound which preferably stabilizes the active ingredient form and ensures a relatively high and also stable absorption of the active ingredient via the skin over a longer period of time.

Various mechanisms for increasing penetration are known, such as a lowering of the melting point of the active pharmaceutical ingredient and/or the reduction of the skin barrier, e.g. by opening tight junctions in the skin.

Penetration enhancers are preferably characterized by at least one of the following properties.

Ideally, the effect of penetration enhancers is fast, and the activity and duration of the effect should be both predictable and reproducible.

Penetration enhancers should not have any pharmacological activity within the body, i.e. they should not bind to any receptor sites, for example.

Penetration enhancers preferably should act unidirectionally, i.e. they should allow therapeutic active ingredients to penetrate the body and at the same time prevent the loss of endogenous material from the body.

When penetration enhancers are removed from the skin, the barrier properties should return both quickly and completely.

Penetration enhancers should be suitable for formulating in different formulations, i.e. they should be compatible with both excipients and drugs.

Penetration enhancers should be cosmetically acceptable and feel good on the skin, be non-toxic, non-irritating and non-allergenic.

The function and properties of penetration enhancers are described, for example, in the publication by Williams et al. “Penetration Enhancers”, Advanced Drug Delivery reviews, 56 (2004), 603 to 618, or in the publication by Amjadi et al. “Recent advances in skin penetration enhancers for transdermal gene and drug delivery”, Current Gene Therapy 17(2), 2017, or in the publication by Gupta et al. “Effect of chemical penetration enhancers on skin permeability: In silico screening using molecular dynamics simulations”, Scientific Reports, 9_1456 (2019), the contents of which are hereby incorporated in full.

A solvent is an agent that dissolves the active ingredient. An important requirement for suitability as a solvent is that neither the dissolving nor the dissolved substance changes chemically during the dissolving process. In contrast to a penetration enhancer, the solvent itself does not necessarily have to increase the permeation of the active ingredient through the patient's skin by reducing the patient's skin barrier.

The transdermal therapeutic system according to the invention is preferably characterized in that the backing layer comprises an elastic woven, knitted, or non-woven fabric. Said fabric is preferably stretchable in at least one direction, preferably in two directions. This refers to the stretchability or elasticity in the longitudinal and/or transverse direction, but not in the thickness direction, of the woven, knitted, or non-woven fabric.

The use of a non-occlusive, longitudinally and/or transversely stretchable woven, knitted, or non-woven fabric as a backing layer is particularly preferred.

Elasticity or being stretchable in at least one, preferably in two (longitudinal and/or transverse) directions, is understood to mean here the ability of the transdermal therapeutic system to stretch in at least one, preferably in two different directions, preferably in the longitudinal and transverse directions, but not in the thickness direction, in relation to the initial state of the material, without the original shape being lost. Permanent deformation of the stretched material does not occur. Elasticity is evaluated by means of elongation, which is specified as a dimensionless number or, when multiplied by 100, as a percentage value. In the transdermal therapeutic system according to the invention, the elongation is preferably 1 to 100%, particularly preferably 10 to 50%, and most preferably 15 to 30%, in relation to the original dimensions of the transdermal therapeutic system. Elasticity is determined in accordance with ISO 13934-1 of Apr. 10, 2013.

The use of such a stretchable woven, knitted, or non-woven fabric has the advantage that the transdermal therapeutic system according to the invention or the active ingredient-containing patch, even in large embodiments and when applied to flexible regions of the body, such as joints of the extremities, is very comfortable to wear, does not restrict mobility, and has a high adhesion to the skin, and thus prevents unintentional detachment.

The transdermal therapeutic system according to the invention is preferably characterized in that the matrix layer is formed as a single layer. In this, the diclofenac, the occlusive adhesive component and the at least one solvent dimethylpropyleneurea are present as a mixture in one and the same layer.

Since the occlusive adhesive component is preferably a hydrophobic adhesive component, a two-phase matrix layer is thus preferably formed, comprising an outer non-polar phase and an inner polar phase, wherein the outer non-polar phase comprises the at least one occlusive adhesive component, and the inner polar phase comprises diclofenac and the at least one solvent dimethylpropyleneurea.

The transdermal therapeutic system according to the invention is preferably characterized in that the matrix layer further comprises a mild acid, preferably with a pks of less than or equal to 5.

The transdermal therapeutic system according to the invention is preferably characterized in that the matrix layer further comprises citric acid and/or dilute hydrochloric acid or another organic acid such as acetic acid, uric acid and/or lactic acid.

The citric acid serves here as a proton donor and has the effect that diclofenac is present in the transdermal therapeutic system according to the invention as a free base.

The transdermal therapeutic system according to the invention is preferably characterized in that the solvent dimethylpropyleneurea is present in the matrix layer in an amount of 0.5 to 20 wt %, particularly preferably in an amount of 2 to 15 wt %, and most preferably in an amount of 5 to 10 wt %, based on the total weight of the matrix layer.

Dimethylpropyleneurea can be utilized alone or in combination with penetration enhancers.

The transdermal therapeutic system according to the invention is preferably characterized in that furthermore at least one penetration enhancer is contained in the matrix layer.

Suitable penetration enhancers comprise fatty acids and/or fatty acid esters, such as pentanoic acid, hexanoic acid, octanoic acid, nonanoic acid, decanoic acid, lauric acid, myristic acid, palmitic acid, stearic acid, arachidic acid, behenic acid, lignoceric acid, isoverlinic acid, neoheptonic acid, neonanonic acid, isostearic acid, oleic acid, palmitoleic acid, linolenic acid, vaccenic acid, petroselinic acid, elaidic acid, oleic acid, arachidonic acid, gadoleic acid, erucic acid, ethyl acetate, methyl propylate, butyl acetate, methyl valerate, diethyl sebacitate, methyl laurate, ethyl oleate, isopropyl decanoate, isopropyl myristate (myristic acid isopropyl ester), isopropyl palmitate and/or isopropyl oleate.

Other suitable penetration enhancers comprise polyhydric alcohols, such as propylene glycol or dipropylene glycol.

Preferably, the transdermal therapeutic system according to the invention contains dimethylpropyleneurea as solvent and a polyhydric alcohol, such as propylene glycol or dipropylene glycol, as penetration enhancer.

The transdermal therapeutic system according to the invention is preferably characterized in that the matrix layer contains at least on penetration enhancer in an amount of 0.5 to 20 wt %, particularly preferably in an amount of 2 to 15 wt %, and most preferably in an amount of 5 to 10 wt % in the matrix layer, based on the total weight of the matrix layer.

The transdermal therapeutic system according to the invention is preferably characterized in that the matrix layer contains propyleneurea in an amount of 0.5 to 20 wt %, particularly preferably in an amount of 2 to 15 wt %, and most preferably in an amount of 5 to 10 wt % in the matrix layer, based on the total weight of the matrix layer. Propyleneurea serves as the preferred solvent here.

Furthermore, the transdermal therapeutic system according to the invention is characterized in that no penetration enhancers from the class of pyrrolidones, more particularly N-methyl-2-pyrrolidone, sulfoxides, more particularly dimethyl sulfoxide (DMSO), formamides, more particularly dimethylformamide (DMF), and/or 1-dodecylazacycloheptan-2-one or laurocapram (azone) and/or derivatives are present in the transdermal therapeutic system according to the invention.

The transdermal therapeutic system according to the invention is preferably characterized in that the citric acid is present in the matrix layer in an amount of 0.1 to 10 mol, preferably 0.3 to 5 mol, particularly preferably 0.3 to 2 mol, per mol of diclofenac.

It is particularly preferred that the occlusive adhesive components are non-polar polymers.

The transdermal therapeutic system according to the invention is preferably characterized in that the occlusive adhesive component comprises a polyisobutylene adhesive, preferably a polyisobutylene adhesive based on a low molecular weight polyisobutylene and a high molecular weight polyisobutylene, and/or a styrene-isoprene-styrene block copolymer.

The transdermal therapeutic system according to the invention is preferably characterized in that the matrix layer contains a low molecular weight polyisobutylene as an occlusive adhesive component in an amount of 30 to 80 wt %, preferably 45 to 60 wt %, based on the weight of the matrix layer.

The transdermal therapeutic system according to the invention is preferably characterized in that the matrix layer contains a high molecular weight polyisobutylene as an occlusive adhesive component in an amount of 10 to 30 wt %, preferably 15 to 25 wt %, based on the weight of the matrix layer.

The transdermal therapeutic system according to the invention is preferably characterized in that the matrix layer contains a total amount of polyisobutylene as an occlusive adhesive component in an amount of 45 to 95 wt %, preferably 55 to 85 wt %, particularly preferably 65 to 75 wt %, more particularly 68 to 72 wt %, based on the weight of the matrix layer The transdermal therapeutic system according to the invention is preferably characterized in that the occlusive adhesive component a polyisobutylene adhesive based on a mixture comprising a low molecular weight polyisobutylene and a high molecular weight polyisobutylene in a ratio of 50 to 95 to 50 to 5.

The low molecular weight polyisobutylene, as described above, preferably is a polyisobutylene with an average molecular weight (determined from viscosity measurements) of 20,000 to 60,000 g/mol, more particularly 40,000 g/mol (determined, for example, by gel permeation chromatography).

The low molecular weight polyisobutylene, as described above, preferably is a polyisobutylene with a limiting viscosity of 27.5 to 31.2 cm³/g.

An example of a commercially available, suitable low molecular weight polyisobutylene is available under the trade name Oppanol B10 from BASF.

The high molecular weight polyisobutylene, as described above, preferably is a polyisobutylene with an average molecular weight (determined from viscosity measurements) of 1,000,000 to 1,200,000 g/mol, more particularly 1,110,000 g/mol (determined, for example, by gel permeation chromatography).

The high molecular weight as described above, preferably is a polyisobutylene with a limiting viscosity of 416 to 479 cm³/g.

An example of a commercially available, suitable high molecular weight polyisobutylene is available under the trade name Oppanol B100, which is also known under the trade name Oppanol N100 from BASF.

Preferably, the occlusive adhesive component contains, in addition to the low molecular weight and/or the high molecular weight polyisobutylene, from 0 to 15 wt % (the value 0 being just excluded), preferably from 3 to 8 wt %, of at least one styrene-isoprene-styrene block copolymer.

The transdermal therapeutic system according to the invention is preferably characterized in that diclofenac is contained in the matrix layer in an amount of 0.5 to 10 wt %, preferably 2 to 5 wt %, based on the weight of the matrix layer.

The transdermal therapeutic system according to the invention is preferably characterized in that solely dimethylpropyleneurea is present as a solvent.

Most preferably, dimethylpropylurea is present in the matrix layer as the only solvent in combination with citric acid and/or hydrochloric acid.

A combination of dimethylenepropylurea as a solvent and a polyhydric alcohol such as propylene glycol as a penetration enhancer in the presence or absence of citric acid and/or hydrochloric acid is also very suitable.

In another embodiment, the transdermal therapeutic system for administering diclofenac is preferably characterized in that it comprises a backing layer and a matrix layer, wherein the matrix layer contains diclofenac, at least one occlusive adhesive component and at least one penetration enhancer, wherein the at least one penetration enhancer comprises a polyhydric alcohol, such as propylene glycol. In this case, no solvent such as dimethylenepropylurea is contained.

Otherwise, all definitions as described above and below apply to this alternative embodiment.

In principle, all pharmaceutically acceptable salts and solvates of diclofenac can be used.

However, the transdermal therapeutic system according to the invention is preferably characterized in that the diclofenac is present as a diclofenac sodium salt.

In another preferred embodiment, the transdermal therapeutic system according to the invention contains at least one antioxidant, preferably in the matrix layer.

The at least one antioxidant is preferably selected from alpha-tocopherol, ascorbyl palmitate, and butylhydroxytoluene. The at least one antioxidant is preferably present in the matrix layer in an amount of 0.05 to 1.5 wt %, preferably 0.2 to 1 wt %, based on the total weight of the matrix layer.

The use of an antioxidant has the advantage that the transdermal therapeutic system according to the invention remains stable over a longer period of time and under various external conditions.

The transdermal therapeutic system according to the invention is further preferably characterized in that the matrix layer has a basis weight of 50 to 400 g/m², preferably 70 to 150 g/m², and particularly preferably 90 to 120 g/m².

In another preferred embodiment, the transdermal therapeutic system according to the invention is characterized in that it comprises a surface area of about 20 to 250 cm², preferably of about 50 to about 150 cm².

Furthermore, in another preferred embodiment, the transdermal therapeutic system according to the invention is characterized in that it comprises a removable protective layer, preferably of a siliconized polyethylene terephthalate film, which is adhered to the side of the matrix layer which is not the backing layer. This removable protective layer (by siliconization on the side with which the protective layer is in contact with the matrix) makes packaging and transport of the transdermal therapeutic system according to the invention easier. The present invention further relates to a transdermal therapeutic system as described above for use as a medicinal product.

Furthermore, the present invention relates to a transdermal therapeutic system as described above for use as a medicinal product in the treatment of pain and inflammatory conditions, such as inflammatory rheumatic diseases, such as chronic polyarthritis, fibromyalgia, or osteoarthritis; acute attacks of gout; joint injuries during sports; pain and swelling after surgery; herniated discs; and venous disorders.

The invention is explained below with reference to non-limiting examples.

### EXAMPLES

The following transdermal therapeutic systems were manufactured according to standard procedures. The tested systems had compositions according to the following Table 1. The quantities are to be understood as % by weight in relation to the total weight of the matrix layer.

TABLE 1

| Formulation | A | B | C | D | E | F |
|---|---|---|---|---|---|---|
| Diclofenac Na | 3.70 | 3.70 | 3.70 | 3.70 | 3.70 | 3.70 |
| Oleic acid | 10.00 | — | — | — | — | — |
| Propylene glycol | — | 10.00 | 7.40 | 7.40 | — | — |
| Propylene urea | — | — | — | — | 7.40 | 7.40 |
| Kollidon 90F | 7.50 | 7.50 | — | — | — | — |
| Hydrobrite HV | 3.80 | 3.80 | 4.90 | 6.50 | 6.10 | 6.10 |
| Oppanol B10 | 50.60 | 50.60 | 50.60 | 50.60 | 50.60 | 50.60 |
| Oppanol B100 | 16.90 | 16.90 | 16.90 | 16.90 | 19.60 | 19.60 |
| SIS 5229P | 7.50 | 7.50 | 7.50 | 7.50 | 7.50 | 7.50 |
| Dimethylpropyleneurea | — | — | 7.40 | 7.40 | 7.40 | 7.4 |
| HCl | — | — | — | — | 0.40 | 0.40 |
| Citric acid | — | — | 1.60 | — | — | — |

Kollidon 90F: polyvinylpyrrolidone copolymer
Hydrobrite HV: mineral oil
Oppanol B10: low molecular weight polyisobutylene (BASF)
Oppanol B100: high molecular weight polyisobutylene (BASF)
SIS 5229P: styrene-isoprene-styrene block copolymer (JSR Corporation)

A transdermal therapeutic system as disclosed in EP 1 312 360 A1 (Dojin patch) with 1.00 wt % diclofenac Na served as a comparison.

The designations Dojin 001, Dojin 002 and Dojin 003 refer to three Dojin patches from different batches.

As a further comparison, the following system (G) was used. The quantities are to be understood as % by weight in relation to the total weight of the matrix layer.

TABLE 2

| Formulation | G |
|---|---|
| Diclofenac Na | 6.00 |
| DuroTak 387-2287 | 3.50 |
| Kollidon 90F | 4.00 |
| Oleic acid | 10.00 |
| alpha-Tocopherol | 1.00 |
| Eucalyptus Sting | 0.50 |
| Oppanol B10/B100 | 75.00 |

DuroTak 387-2287: acrylate copolymer based on ethyl acetate with free OH groups, without crosslinking agent (non-occlusive adhesive component)

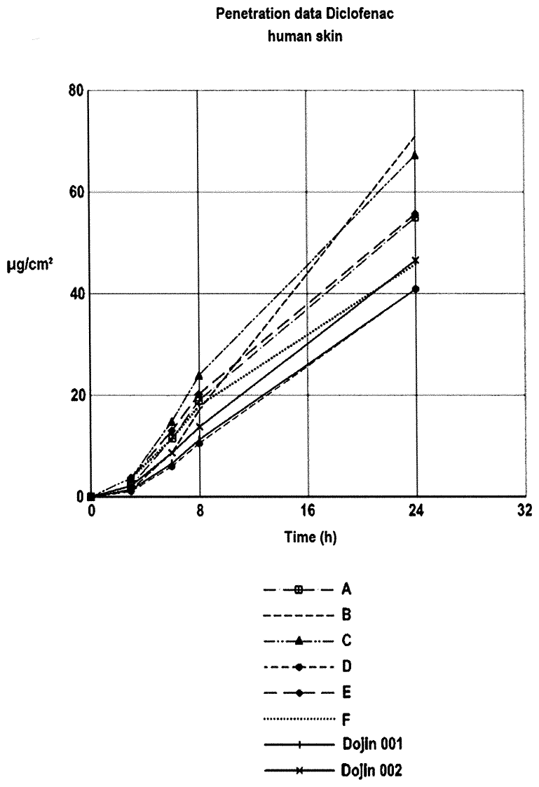

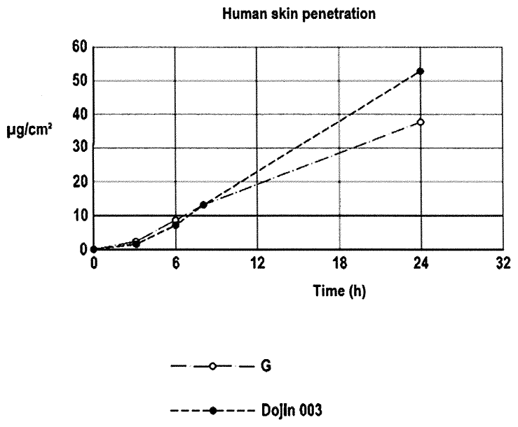

The in vitro human skin permeation of the systems listed in Table 1 and 2 and of Dojin patches 1 and 2 was measured using a Franz cell. The donor compartment contains the substance or formulation (e.g. gels, ointments, solutions, patches). The acceptor compartment is filled with buffer or other solutions. By frequently taking samples from the acceptor compartment, the penetration of a substance through the skin can be monitored over the selected period of time. The influence of penetration enhancers on the penetration of a substance can also be tested using this system. The use of the Franz cell as a diffusion model is particularly suitable for predicting the transport of drugs through human skin (=penetration), which corresponds to systemic availability. However, it is important to note that there is no in vitro-in vivo correlation. Here, the Franz cell was loaded with human abdominal skin obtained from operations. Here, 500 μm of dermatomized skin with a diffusion area of 1.172 cm² was incubated with the topical therapeutic system. A phosphate buffer+0.1% NaN₃ (pH=5.5) with a filling volume of 10 mL was used as the acceptor medium. The penetration measurement was carried out at a temperature of 32° C. The measurement results are shown in FIG. 1 and FIG. 2.

1. A transdermal therapeutic system for administrating diclofenac comprising a backing layer and a matrix layer, wherein the matrix layer contains diclofenac, at least one occlusive adhesive component and at least one solvent, characterized in that the at least one solvent comprises dimethylpropyleneurea.

2. The transdermal therapeutic system according to claim 1, characterized in that the backing layer comprises an elastic woven, knitted, or non-woven fabric.

3. The transdermal therapeutic system according to claim 1, characterized in that furthermore at least one penetration enhancer is contained in the matrix layer.

4. The transdermal therapeutic system according to claim 1, characterized in that the matrix layer comprises citric acid and/or hydrochloric acid.

5. The transdermal therapeutic system according to claim 1, characterized in that dimethylpropyleneurea is contained in the matrix layer in an amount of 0.5 to 20 wt %, based on the total weight of the matrix layer.

6. The transdermal therapeutic system according to claim 1, characterized in that citric acid and/or hydrochloric acid is present in the matrix layer in an amount of 0.1 to 10 mol per mol of diclofenac.

7. The transdermal therapeutic system according to claim 1, characterized in that the occlusive adhesive component comprises a polyisobutylene adhesive and/or a styrene-isoprene-styrene block copolymer.

8. The transdermal therapeutic system according to claim 1, characterized in that the matrix layer contains a low molecular weight polyisobutylene as an occlusive adhesive component in an amount of 30 to 80 wt % based on the weight of the matrix layer.

9. The transdermal therapeutic system according to claim 1, characterized in that the matrix layer contains a high molecular weight polyisobutylene as an occlusive adhesive component in an amount of 10 to 30 wt % based on the weight of the matrix layer.

10. The transdermal therapeutic system according to claim 1, characterized in that the occlusive adhesive component contains a polyisobutylene adhesive based on a mixture comprising a low molecular weight polyisobutylene and a high molecular weight polyisobutylene in a ratio of 50 to 95 to 50 to 5.

11. The transdermal therapeutic system according to claim 1, characterized in that the diclofenac is present in the matrix layer in an amount of 0.5 to 10 wt %, based on the weight of the matrix layer.

12. The transdermal therapeutic system according to claim 1, characterized in that the diclofenac is present as a diclofenac sodium salt.

13. The transdermal therapeutic system according to claim 1, characterized in that solely dimethylpropyleneurea is present as solvent, more particularly in combination with citric acid, in the matrix layer.

14. A medicinal product comprising the transdermal therapeutic system according to claim 1.

15. A method for the treatment of pain and inflammatory conditions comprising the administration of the transdermal therapeutic system of claim 1.

16. The transdermal therapeutic system according to claim 1, characterized in that a polyhydric alcohol penetration enhancer is contained in the matrix layer.

17. The transdermal therapeutic system according to claim 1, characterized in that citric acid and/or hydrochloric acid is present in the matrix layer in an amount of 0.3 to 2 mol per mol of diclofenac.

18. The transdermal therapeutic system according to claim 7, characterized in that the polyisobutylene adhesive is comprised of a low molecular weight polyisobutylene and a high molecular weight polyisobutylene.

19. The transdermal therapeutic system according to claim 1, characterized in that the matrix layer contains a low molecular weight polyisobutylene as an occlusive adhesive component in an amount of 45 to 60 wt % based on the weight of the matrix layer.

20. The transdermal therapeutic system according to claim 1, characterized in that the matrix layer contains a high molecular weight polyisobutylene as an occlusive adhesive component in an amount of 15 to 25 wt % based on the weight of the matrix layer.
